(19) United States
(12) Patent Application Publication
(10) Pub. No.: US 2022/0031571 A1
(43) Pub. Date: Feb. 3, 2022

(54) INTELLIGENT PILL DISPENSING DEVICE

(71) Applicant: DLL Solutions Engement Company, Inc., Topsfield, MA (US)

(72) Inventors: James George Clements, Brentwood, NH (US); Kenneth Paul Dzierzek, Topsfield, MA (US)

(73) Assignee: DLL Solutions Engement Company, Inc., Topsfield, MA (US)

(21) Appl. No.: 16/941,080

(22) Filed: Jul. 28, 2020

(51) Int. Cl. A61J 7/04; G16H 20/13; G16H 40/20; G05B 19/4155; A61J 7/00

(52) U.S. Cl. CPC A61J 7/0436; G16H 20/13; G16H 40/20; G06K 9/00087; A61J 7/0076; A61J 7/0481; G05B 2219/40063; G05B 19/4155

(57) ABSTRACT

Techniques are provided for intelligent and secure dispensing of medication to a patient. A methodology implementing the techniques according to an embodiment includes receiving a pill vial into a receptacle of a base unit of a pill dispensing device. The method also includes engaging a locking mechanism to secure the pill vial in the base unit until a release condition is satisfied. The method further includes scanning a label on the pill vial to record a prescription identifier and a dosage schedule, and to register the prescription for usage tracking. The method further includes monitoring patient usage of the prescription based on a count of removals of the pill vial from the receptacle and a timing of the removals and alerting the patient of a missed dose based on the dosage schedule and on detection of an elapsed time since a previous removal of the pill vial from the receptacle.

### BACKGROUND

Many people take prescription medications and supplements on a daily basis. Such medications and supplements are typically dispensed as pills or capsules, in a labeled vial that provides precautionary warnings and instructions as to dosage and refills. It can be difficult, however, for patients to consistently follow the instructions, particularly if they are taking multiple medications, each with a potentially different dosing schedule, which can sometimes result in confusion and either missed dosages or too many dosages. Additionally, some patients suffer from poor vision, memory loss, cognitive impairments, or other such conditions that can exacerbate the problem. In some cases, failure to strictly follow prescribing instructions can lead to challenges in medical treatment, serious medical problems, and even fatalities.

### BRIEF DESCRIPTION OF THE DRAWINGS

FIG. 1 is a perspective view of an intelligent pill dispensing device in an open configuration, in accordance with an embodiment of the present disclosure.

FIG. 2 is a perspective view of the intelligent pill dispensing device in a closed configuration, in accordance with an embodiment of the present disclosure.

FIG. 3 is a perspective view of the intelligent pill dispensing illustrating a user interface, configured in accordance with an embodiment of the present disclosure.

FIG. 4 is a cross sectional view of the intelligent pill dispensing device, configured in accordance with an embodiment of the present disclosure.

FIG. 5 is a block diagram of the processing system of the intelligent pill dispensing device, configured in accordance with an embodiment of the present disclosure.

FIG. 6 is a flowchart illustrating a methodology for operation of the intelligent pill dispensing device, in accordance with an embodiment of the present disclosure.

FIG. 7 is a block diagram schematically illustrating a computing platform configured to host the processing system of the intelligent pill dispensing device, in accordance with an embodiment of the present disclosure.

Although the following Detailed Description will proceed with reference being made to illustrative embodiments, many alternatives, modifications, and variations thereof will be apparent in light of this disclosure.

### DETAILED DESCRIPTION

As previously noted, it can be difficult for patients to follow the prescribing instructions on their medications with consistency and accuracy. This is particularly true if they are taking multiple medications, each with a different dosing schedule, which can result in missed dosages or excessive dosages. Additionally, some patients suffer from poor vision, memory loss, cognitive impairments, or other such conditions that can exacerbate the problem. This can pose a significant problem because, in some cases, failure to strictly follow prescribing instructions can lead to serious medical problems and even fatalities. In still other cases, access to medication may need to be restricted to certain times and dosages, to prevent potential abuse or harm (such as in the case of addiction or dangerous medication that must be taken on a strict schedule to avoid toxicity or ineffective otherwise unintended dosing). To this end, techniques are provided for intelligent dispensing of medication in an efficient and secure manner that can improve patient compliance and safety while enhancing the timeliness and accuracy of information provided to medical professionals.

According to an embodiment, an intelligent pill dispensing device includes a base unit that provides receptacles configured for insertion of pill vials. The medication may be, for example, in the form of pills, capsules, or any other physical form of medication (e.g., ointment, liquid, a pre-loaded syringe, etc.) that are packaged or otherwise containable in a vial. The dispensing device is further configured to scan the label on the vial to determine dosing information and other relevant data, as will be described in greater detail below. Based on this acquired information, the device may further secure the vials and manage the dosing schedule through a system of monitoring activities and alerts, as will also be described below.

In an example use case, a patient receives medication from a pharmacy or doctor and the prescription instructs the patient that one pill should be taken each day. The vial of pills is inserted into a receptacle in the base unit of the pill dispensing device. Note that this placement can be done, for instance, by the patient or a caregiver (e.g., physician, nurse, pharmacist, etc.). The caregiver or patient decides on a morning dose at approximately 8 AM. The pill dispensing device is programmed to allow for the dispensing of one pill per day during a window of time around 8 AM. The programming may occur through a user interface on the device, or through scanning of the prescription label. The device confirms that 8 AM is the desired dosing time, and a locking mechanism covers or otherwise secures the vial to inhibit removal from the receptacle. At approximately 8 AM, the device releases the locking mechanism and illuminates a green colored indicator light. The device will then wait for a selected length of time (based on pharmaceutical best practices), for example 30 minutes, and then cause the indicator light to blink and/or provide an audible alarm or prompt (e.g., “it is time to take your medication”). After an additional appropriate length of time has elapsed, if the pill vial has not been temporarily removed, the device will send a signal (e.g., SMS message, email, voicemail, or other message) to the patient or caregiver or other designated party, indicating that the pill was not taken at the appropriate time. Likewise, if the vial was removed but not reinserted after an appropriate time, for example, two minutes, another audible alarm or electronic notification may be provided. Additionally, if the vial that is reinserted differs from the vial that was removed (e.g., as detected by a confirmatory label scan), the device will recognize this fact and take appropriate action such as providing an alert.

In another example use case, a medication is prescribed, which requires that the patient take the pills at periodic intervals that cannot be less than four hours apart. This information is programmed into the device. Upon removal and reinsertion of the pill vial into the receptacle, the vial is locked, and a red indicator light is illuminated to signal that additional doses of the medication should not be taken. After four hours has elapsed, the red indicator light is turned off and a green indicator light is switched on to indicate that a second dose can be taken when desired. Subsequent removal and replacement of the vial will reset the 4 hour time period, again locking the vial into the receptacle to prevent a potentially confused patient from inadvertently reducing the time between self-administered medications. In some embodiments, in addition to waiting for the appropriate elapsed time, a user identification may be required before the vial is unlocked, to reduce the possibility that a dangerous medicine could be obtained by an unintended person.

In still another example use case, a medication is prescribed, which requires that the patient take three pills per day of equally spaced intervals. Additionally, the prescription suggests that if a dose is missed it should not be made-up. Instead, the patient should maintain normal dosing schedule going forward, skipping the missed dose. The vial is inserted into the receptacle, and the device is programmed for dosing at 7 PM, 1 PM, and 8 PM. At the appropriate time, the green indicator light is illuminated to indicate that a dough should be taken. The patient is then identified with entry of an ID code, a fingerprint scan, or through facial recognition or other biometric, and if successful, the locking mechanism is released. The patient then lifts the vial out of the receptacle, removes the pill, and re-inserts the vial back into the receptacle. The device records that the vial was removed and the time that it was removed and re-engages the locking mechanism to secure the vial. At the second dosing time (in this example, 1 PM) the green indicator light illuminates, but the patient does not attempt to take a dose. After an appropriate time, for example, one hour, the device may sound an audible alarm, flash a blinking light, and/or alert a designated third-party of the missed dose. After an additional time, for example, four hours, the device decides that the 1 PM dose was missed and resets the alarms and begins a timer countdown to the next dose at 8 PM.

In still another example use case, a medication is prescribed, which requires 4 doses per day, for example at 6 AM, 11 AM, 4 PM, and 9 PM. This information is programmed into the device, as described previously. In this example use case, the device is not configured to provide a locking mechanism for the pill vials, and thus the patient is not prevented from removing the pill vials at their discretion. At 6 AM a green indicator light turns on, but if the vial is not removed within a selected reasonable length of time, the indicator converts to a blinking light, an alternate color, and/or an audible alarm is sounded. The duration of these alerting events may be adjusted based on the needs of the patient. In the case of an antibiotic, where a missed dose should not be doubled up at a later time, the device resets and turns on a green indicator light as the subsequent 11 AM dosing time approaches. The device records the event and provide an electronic notification to a designated responsible party, if so programmed. The device may also optionally display the fact that a dose was missed.

In still another example use case, the device will monitor and track the usage and compliance of the patient based on the prescription information that is scanned from the vial label or entered through the user interface. If abnormal usage patterns, noncompliance, or failed identification attempts are detected, an alert may be sent to a designated party. In some embodiments, the need for a refill of the medication may be detected and communicated back to the pharmacy or the doctor through a secure network connection.

In more detail, and according to an embodiment, the intelligent pill dispensing device is configured to monitor usage and assist the patient with the administration of prescribed medicines to reduce the possibility of patient confusion, missed doses, and overdoses. The disclosed techniques can be implemented, for example, in a computing system or a software product executable or otherwise controllable by such systems, although other embodiments will be apparent. In one such embodiment, a methodology implementing the techniques includes receiving a pill vial into a receptacle of a base unit of the pill dispensing device. The method also includes engaging a locking or securing mechanism to secure the pill vial in the base unit until a release condition is satisfied. The method further includes scanning a label on the pill vial to record a prescription identifier and a dosage schedule, and to register the prescription for usage tracking. The method further includes monitoring patient usage of the prescription based on a count of removals of the pill vial from the receptacle and a timing of the removals and alerting the patient of a missed dose based on the dosage schedule and on detection of an elapsed time since a previous removal of the pill vial from the receptacle.

As will be appreciated, the techniques described herein may provide an improved process for dispensing of pills in an efficient manner (the patient simply inserts the vials into a receptacle), compared to existing techniques that require the patient to sort out medicines into pill containers with divided pockets for each day of the week, a process that can be tedious and error prone, and provides no measure of security. These techniques may further be implemented in a combination of hardware and software.

System Architecture

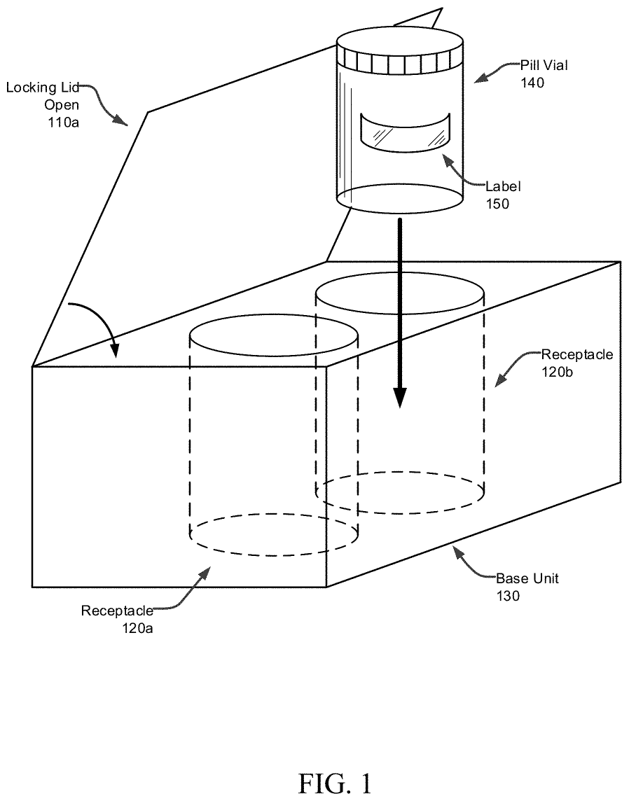

FIG. 1 is a perspective view of an intelligent pill dispensing device in an open configuration 100a, in accordance with an embodiment of the present disclosure. The pill dispensing device is shown to include a base unit 130, one or more receptacles 120a, 120b, and a locking lid 110, shown in the open position 110a. In some embodiments, the base unit 130 may be an appliance-sized device that can be placed conveniently on a bathroom countertop or nightstand. The receptacles 120 are configured to receive a pill vial 140 containing the patient's medication. A label 150 is also shown disposed on the pill vial 140. The label may include printed information that provides an identifying number for the prescription that is useful for ordering refills, dosing information (e.g., the number of pills to be taken and a specified periodic interval), expiration date, and pharmacy contact information, etc. Two receptacles are shown in this illustration, but any number of receptacles may be provided.

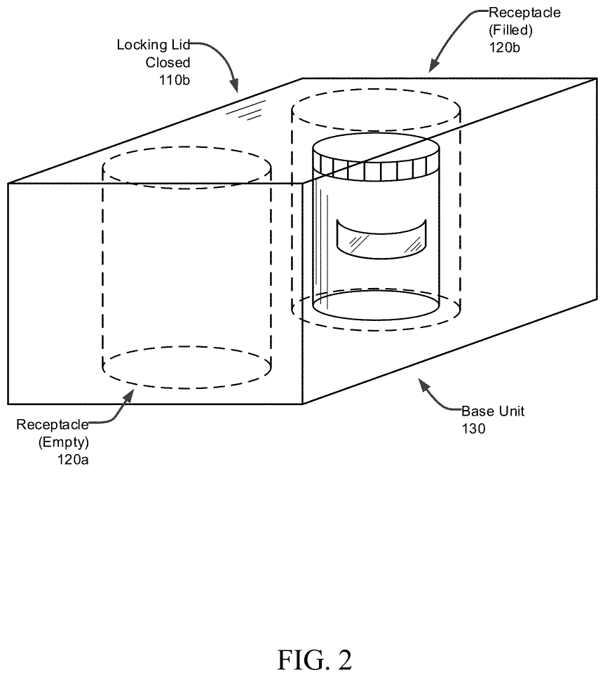

FIG. 2 is a perspective view of the intelligent pill dispensing device in a closed configuration 100b, in accordance with an embodiment of the present disclosure. The pill dispensing device is shown again to include the base unit 130, one or more receptacles 120a, 120b, and the locking lid 110, shown in the close position 110b. In this illustration, the lid 110 is a hinged lid that folds down to cover the receptacle openings and prevent removal of any pill vials stored therein. In some embodiments, other suitable locking mechanisms may be used. For example, the locking lid may slide over the openings, or a clamping device may retain the pill vials and inhibit removal.

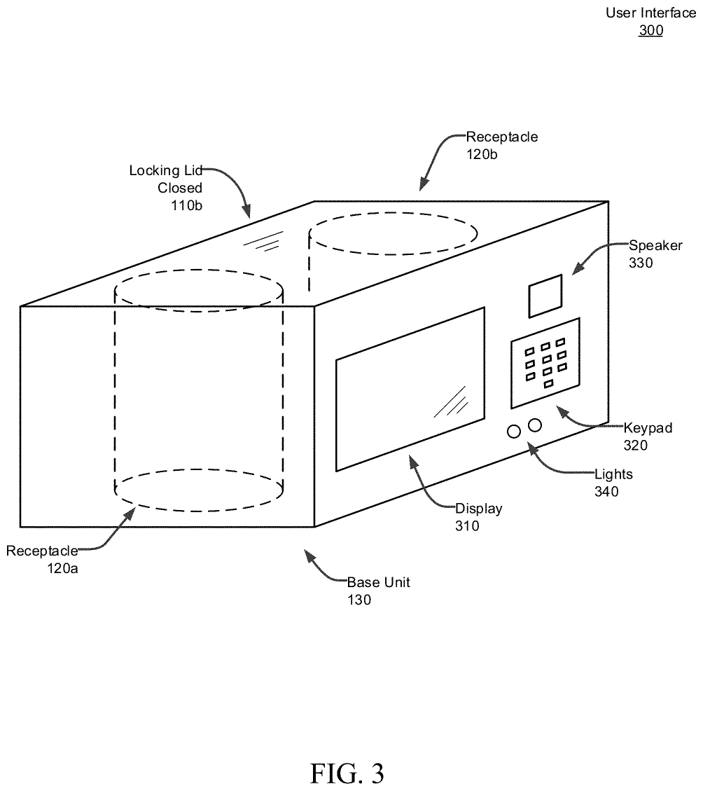

FIG. 3 is a perspective view of the intelligent pill dispensing illustrating a user interface 300, configured in accordance with an embodiment of the present disclosure. The user interface is shown to include a display 310, a keypad 320, a speaker 330, and indicator lights 340. In some embodiments, any combination of these features, or other suitable user interface features (e.g., facial recognition scanner, fingerprint scanner, microphone, etc.), may be provided.

The display 310 is configured to provide information to the patient or other users of the device. Other users may include designated parties responsible for caring for or overseeing the patient. The information may include, for example, a list of the current medications stored in the device, details related to the next upcoming dose (e.g., the time for the dose, the appropriate vial, and the number of pills), warnings about previously missed dosages, and alerts that refills may be needed, to name a few.

The keypad 320 is configured to allow the patient or other users of the device to enter information. This information may include, for example, a personal identification number (PIN) used to verify the identity of the patient prior to allowing access to a pill vial. In some embodiments, the information may also include answers, from the user, to queries from the device. For example, the device may ask the user if a refill is desired, and the user may press a yes or no button in response.

The speaker 330 is configured to provide audible alerts or pre-recorded prompts or instructions to the patient or other users. For example, if the scheduled time for medication dosage has passed and the patient has not attempted to remove the vial, an audible alert may be sounded to notify the patient of this fact. A prerecorded verbal message (e.g., from caregiver or pharmacist can be stored in the system and aurally presented to the patient via speaker 330. Any number of helpful instructions can be provided, as will be appreciated. This will potentially help mitigate the confusion problems noted previously with respect to patients that suffer from poor vision, memory loss, cognitive impairments, or other such conditions. For example, a patient with poor vision may have trouble reading the small print on the prescription label and a patient with cognitive and parents may not be able to read at all. Additionally, the alerts will help to remind the patient that suffers from memory loss, that it is time to take the appropriate medicine.

The indicator lights 340, are configured to provide visual alerts to the patient or other users, for the same purpose as the audible alerts described above. In some embodiments, a green light may indicate a satisfactory condition, while a red light may indicate that some problem has occurred which requires attention. In the case of a red light, more information may be provided by the display.

In some embodiments, a facial recognition scanner and/or fingerprint scanner may be provided and configured as an alternative method for the patient or other users to identify themselves to the device.

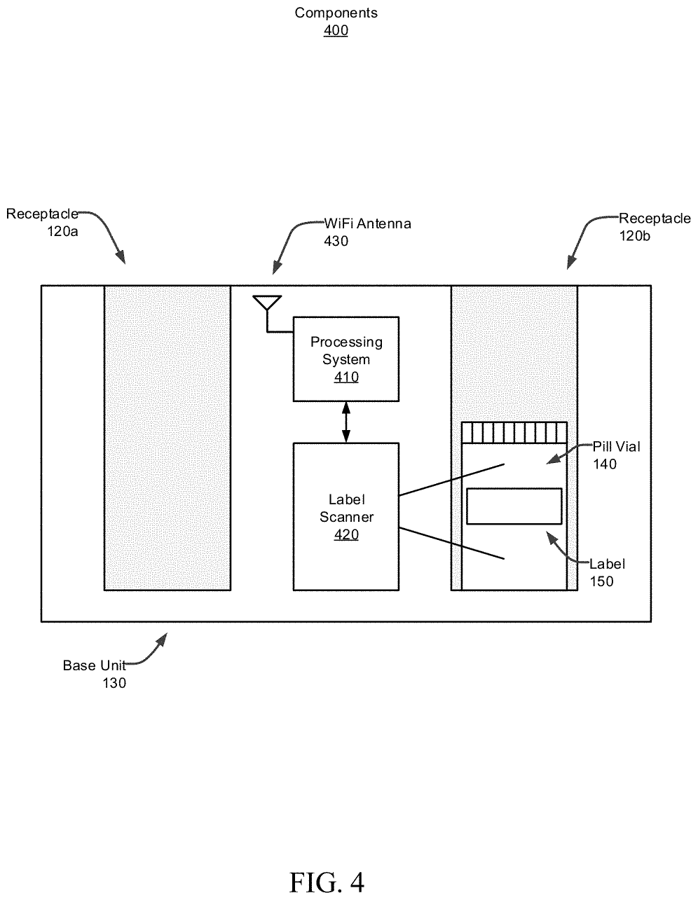

FIG. 4 is a cross sectional view of the intelligent pill dispensing device showing internal components 400, configured in accordance with an embodiment of the present disclosure. The base unit 130 and two receptacles 120a, 120b are shown, with receptacle 120b containing pill vial 140. Label scanner 420 is also shown, along with processing system 410 and Wi-Fi antenna 430. Label scanner 420 is configured to scan the label 150 on the pill vial. Label scanner 420 is configured to provide a digital image of the scanned label to the processing system 410, which may then perform optical character recognition (OCR) on the image to determine the text that is printed on the label. For instance, the label text will typically indicate a prescription identifier (e.g., name of medication and maker) and a dosage schedule (e.g., number and timing of doses), and any other pertinent information such as side effects, whether or not the medication should be taken with food, or other medicines that should be avoided while the given medication is being taken. In some embodiments, a separate label scanner may be provided for each receptacle, or a single label scanner may be configured to scan the contents of multiple receptacles.

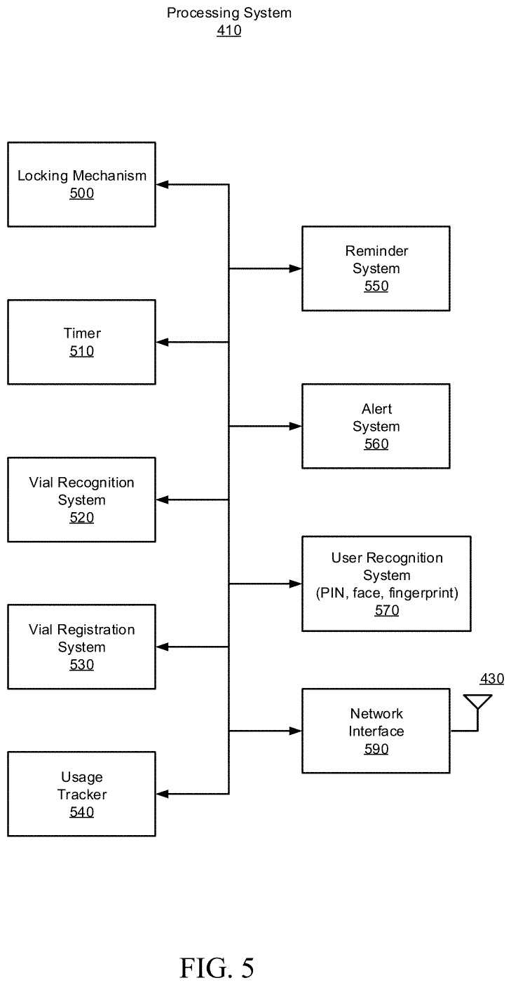

FIG. 5 is a block diagram of the processing system 410 of the intelligent pill dispensing device, configured in accordance with an embodiment of the present disclosure. The processing system 410 is shown to include a locking mechanism 500, a timer 510, a vial recognition system 520, a vial registration system 530, a usage tracker 540, a reminder system 550, an alert system 560, a user recognition system 570, and a network interface 590.

The locking mechanism 500 is configured to secure the receptacles so that any pill vials contained therein are not easily removed. In some embodiments, this may be accomplished by lowering and locking a lid over the base unit to block access to the receptacles. In some embodiments, other suitable techniques may be employed to secure the pill vials in light of the present disclosure, including other access prevention mechanisms and/or vial grasping mechanisms.

The timer 510 is configured to keep track of the current time and date and the time and date of each removal of a pill vial. The timer 510 is also configured to measure the elapsed time between the current time and the previous removal of each pill vial.

The vial recognition system 520 is configured to process the digital image of the pill vial label, provided by the label scanner 420, (e.g., using OCR or other suitable techniques) to recognize and store the information printed on the label 150.

The vial registration system 530 is configured to process the stored label information to register the associated pill vial with the device so that subsequent usage monitoring of that pill vial can be performed. For example, the vial registration system 530 can recognize when a previously registered pill vial is reinserted into a receptacle. In some embodiments, the vial registration system 530 can also detect that a vial has been removed and replaced with a different vial, in which case, appropriate action may be taken (e.g., issuing an alert).

The usage tracker 540 is configured to monitor usage of each pill vial that has been stored in a receptacle of the device and registered with the device. Based on measurements provided by the timer 510, the usage tracker 540 can track the times of usage of each pill vial (e.g., removal of the pill vial from the receptacle) and maintain a count of the doses taken. The monitored usage may then be compared to the prescribing information to determine patient compliance and detect abnormal usage patterns.

The reminder system 550 is configured to provide a reminder to the patient or other designated user that a dosing time is imminent. The reminder may be provided through any or all components of the user interface including display 310, speaker 330, and indicator lights 340. Additionally, since the prescribing information is known from the label scan, and a count of the number of doses taken is monitored by the usage tracker 540, the reminder system 550 may inform the patient or user that a refill should be requested within a specified time.

The alert system 560 is configured to alert the patient or other designated user that a dose has been missed or other abnormal usage patterns have been detected (e.g., based on a count of the number of doses taken as provided by the usage tracker 540). The alert may be provided through any or all components of the user interface including display 310, speaker 330, and indicator lights 340.

The user recognition system 570 is configured to recognize the patient or designated user prior to allowing removal of medications, as a security precaution. The recognition may be based on entry of a PIN through the keypad, facial recognition, and/or fingerprint recognition. Other suitable recognition techniques may also be used, in light of the present disclosure. In some embodiments, a particular PIN may be employed to allow for removal of medications prior to the designated dosing time. For example, the medications may no longer be needed, and should be removed for disposal, or a designated caretaker may need to remove the medications upon instructions from a doctor.

The network interface 590 is configured to provide communication capabilities between the device and other networked entities, including, for example, the Internet. In some embodiments, the communication may be provided through Wi-Fi, on that any suitable communication mechanism may be employed in light of the present disclosure. Networking capabilities may be employed to allow the device to communicate with a pharmacy to request prescription refills, with the patient's doctor, or with other designated responsible parties.

Methodology

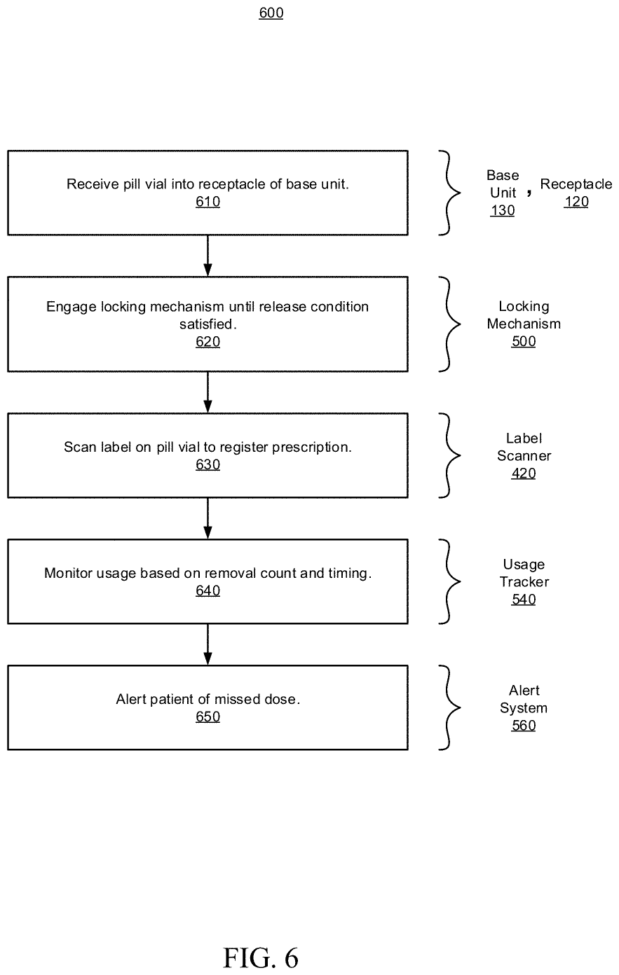

FIG. 6 is a flowchart illustrating a methodology 600 for operation of the intelligent pill dispensing device, in accordance with an embodiment of the present disclosure. As can be seen, the example method includes a number of phases and sub-processes, the sequence of which may vary from one embodiment to another. However, when considered in the aggregate, these phases and sub-processes form a process for pill dispensing, in accordance with certain of the embodiments disclosed herein. These embodiments can be implemented, for example, using the system architecture illustrated in FIGS. 1-5, as described herein. However other system architectures can be used in other embodiments, as will be apparent in light of this disclosure. To this end, the correlation of the various functions shown in FIG. 6 to the specific components illustrated in the other figures is not intended to imply any structural and/or use limitations. Rather, other embodiments may include, for example, varying degrees of integration wherein multiple functionalities are effectively performed by one system. For example, in an alternative embodiment a single module having decoupled sub-modules can be used to perform all of the functions of method 600. Thus, other embodiments may have fewer or more modules and/or sub-modules depending on the granularity of implementation. In still other embodiments, the methodology depicted can be implemented as a computer program product including one or more non-transitory machine-readable mediums that when executed by one or more processors cause the methodology to be carried out. Numerous variations and alternative configurations will be apparent in light of this disclosure.

As illustrated in FIG. 6, in an embodiment, method 600 for dispensing of pills commences at operation 610, by receiving a pill vial into a receptacle of a base unit.

Next, at operation 620, a locking mechanism is engaged to secure the pill vial in the base unit until a release condition is satisfied. In some embodiments, the release condition includes detection of an elapsed time since a previous removal of the pill vial from the receptacle. In some embodiments, the release condition includes one or more of verification of a personal identification number entered by a user of the system, verification of a fingerprint scan of the user of the system, and facial recognition of the user of the system.

At operation 630, a label on the pill vial is scanned to record a prescription identifier and a dosage schedule, and to register the prescription for usage tracking.

At operation 640, patient usage of the prescription is monitored based on a count of removals of the pill vial from the receptacle and a timing of the removals.

At operation 650, the patient is alerted of a missed dose based on the dosage schedule and on the detection of an elapsed time since the previous removal of the pill vial from the receptacle.

Of course, in some embodiments, additional operations may be performed, as previously described in connection with the system. For example, the label on the pill vial may be scanned again, after re-insertion of the pill vial into the receptacle, to recognize and verify the medication based on the registration. In some embodiments, a pharmacy may be alerted that a refill of the prescription is needed based on the monitored patient usage and the dosage schedule, and/or a designated party may be alerted in response to detection of abnormal medication usage based on the monitored patient usage and the dosage schedule. In some embodiments, the user of the device may be the patient, the designated party, a pharmacist, or a prescribing doctor.

Example System

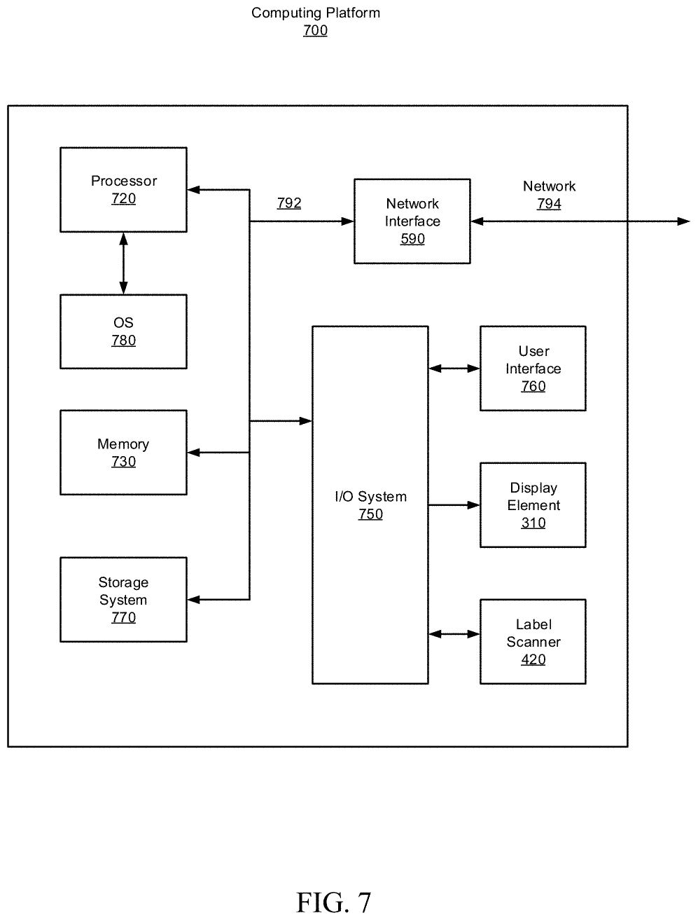

FIG. 7 is a block diagram schematically illustrating a computing platform 700 configured to host the processing system of the intelligent pill dispensing device, in accordance with an embodiment of the present disclosure. In some embodiments, platform 700 may be hosted on, or otherwise incorporated into a portable computer, a smart device, an embedded system, or any suitable computing or communication platform. Any combination of different devices may be used in certain embodiments.

In some embodiments, platform 700 may comprise any combination of a processor 720, a memory 730, a network interface 590, an input/output (I/O) system 750, a user interface 760, a display element 310, a storage system 770, and a label scanner 420. As can be further seen, a bus and/or interconnect 792 is also provided to allow for communication between the various components listed above and/or other components not shown. Platform 700 can be coupled to a network 794 through network interface 590 to allow for communications with other computing devices, platforms, devices to be controlled, or other resources. Other componentry and functionality not reflected in the block diagram of FIG. 7 will be apparent in light of this disclosure, and it will be appreciated that other embodiments are not limited to any particular hardware configuration.

Processor 720 can be any suitable processor, and may include one or more coprocessors or controllers, such as an audio processor, a graphics processing unit, or hardware accelerator, to assist in control and processing operations associated with platform 700. In some embodiments, the processor 720 may be implemented as any number of processor cores. The processor (or processor cores) may be any type of processor, such as, for example, a micro-processor, an embedded processor, a digital signal processor (DSP), a graphics processor (GPU), a tensor processing unit (TPU), a network processor, a field programmable gate array or other device configured to execute code. The processors may be multithreaded cores in that they may include more than one hardware thread context (or “logical processor”) per core. Processor 720 may be implemented as a complex instruction set computer (CISC) or a reduced instruction set computer (RISC) processor. In some embodiments, processor 720 may be configured as an x86 instruction set compatible processor.

Memory 730 can be implemented using any suitable type of digital storage including, for example, flash memory and/or random-access memory (RAM). In some embodiments, the memory 730 may include various layers of memory hierarchy and/or memory caches as are known to those of skill in the art. Memory 730 may be implemented as a volatile memory device such as, but not limited to, a RAM, dynamic RAM (DRAM), or static RAM (SRAM) device. Storage system 770 may be implemented as a non-volatile storage device such as, but not limited to, one or more of a hard disk drive (HDD), a solid-state drive (SSD), a universal serial bus (USB) drive, an optical disk drive, tape drive, an internal storage device, an attached storage device, flash memory, battery backed-up synchronous DRAM (SDRAM), and/or a network accessible storage device. In some embodiments, storage 770 may comprise technology to increase the storage performance enhanced protection for valuable digital media when multiple hard drives are included.

Processor 720 may be configured to execute an Operating System (OS) 780 which may comprise any suitable operating system, such as Google Android (Google Inc., Mountain View, Calif.), Microsoft Windows (Microsoft Corp., Redmond, Wash.), Apple OS X (Apple Inc., Cupertino, Calif.), Linux, or a real-time operating system (RTOS). As will be appreciated in light of this disclosure, the techniques provided herein can be implemented without regard to the particular operating system provided in conjunction with platform 700, and therefore may also be implemented using any suitable existing or subsequently developed platform.

Network interface circuit 590 can be any appropriate network chip or chipset which allows for wired and/or wireless connection between other components of platform 700 and/or network 794, thereby enabling platform 700 to communicate with other local and/or remote computing systems, servers, cloud-based servers, and/or other resources. Wired communication may conform to existing (or yet to be developed) standards, such as, for example, Ethernet. Wireless communication may conform to existing (or yet to be developed) standards, such as, for example, cellular communications including LTE (Long Term Evolution) and 5G, Wireless Fidelity (Wi-Fi), Bluetooth, and/or Near Field Communication (NFC). Exemplary wireless networks include, but are not limited to, wireless local area networks, wireless personal area networks, wireless metropolitan area networks, cellular networks, and satellite networks.

I/O system 750 may be configured to interface between various I/O devices and other components of platform 700. I/O devices may include, but not be limited to, user interface 760 and display element 310. User interface 760 may include devices (not shown in this figure) such as a keypad, touchpad, speaker, microphone, keyboard, mouse, etc. User interface 760 may include identification devices such as a fingerprint scanning device, a facial recognition device, and the like. I/O system 750 may include a graphics subsystem configured to perform processing of images for rendering on the display element. Graphics subsystem may be a graphics processing unit or a visual processing unit (VPU), for example. An analog or digital interface may be used to communicatively couple graphics subsystem and the display element. For example, the interface may be any of a high definition multimedia interface (HDMI), DisplayPort, wireless HDMI, and/or any other suitable interface using wireless high definition compliant techniques. In some embodiments, the graphics subsystem could be integrated into processor 720 or any chipset of platform 700.

It will be appreciated that in some embodiments, the various components of platform 700 may be combined or integrated in a system-on-a-chip (SoC) architecture. In some embodiments, the components may be hardware components, firmware components, software components or any suitable combination of hardware, firmware or software.

Computing platform 700 is configured to perform the pill dispensing functions, as described previously, including operation of the processing system circuits/components illustrated in FIG. 5, as described above. These components can be implemented or otherwise used in conjunction with a variety of suitable software and/or hardware that is coupled to or that otherwise forms a part of platform 700. These components can additionally or alternatively be implemented or otherwise used in conjunction with user I/O devices that are capable of providing information to, and receiving information and commands from, a user.

In some embodiments, these circuits may be installed local to platform 700, as shown in the example embodiment of FIG. 7. Alternatively, platform 700 can be implemented in a client-server arrangement wherein at least some functionality associated with these circuits is provided to platform 700 using an applet, such as a JavaScript applet, or other downloadable module or set of sub-modules. Such remotely accessible modules or sub-modules can be provisioned in real-time, in response to a request from a client computing system for access to a given server having resources that are of interest to the user of the client computing system. In such embodiments, the server can be local to network 794 or remotely coupled to network 794 by one or more other networks and/or communication channels. In some cases, access to resources on a given network or computing system may require credentials such as usernames, passwords, and/or compliance with any other suitable security mechanism.

In various embodiments, platform 700 may be implemented as a wireless system, a wired system, or a combination of both. When implemented as a wireless system, platform 700 may include components and interfaces suitable for communicating over a wireless shared media, such as one or more antennae, transmitters, receivers, transceivers, amplifiers, filters, control logic, and so forth. An example of wireless shared media may include portions of a wireless spectrum, such as the radio frequency spectrum and so forth. When implemented as a wired system, platform 700 may include components and interfaces suitable for communicating over wired communications media, such as input/output adapters, physical connectors to connect the input/output adaptor with a corresponding wired communications medium, a network interface card (NIC), disc controller, video controller, audio controller, and so forth. Examples of wired communications media may include a wire, cable metal leads, printed circuit board (PCB), backplane, switch fabric, semiconductor material, twisted pair wire, coaxial cable, fiber optics, and so forth.

Various embodiments may be implemented using hardware elements, software elements, or a combination of both. Examples of hardware elements may include processors, microprocessors, circuits, circuit elements (for example, transistors, resistors, capacitors, inductors, and so forth), integrated circuits, ASICs, programmable logic devices, digital signal processors, FPGAs, logic gates, registers, semiconductor devices, chips, microchips, chipsets, and so forth. Examples of software may include software components, programs, applications, computer programs, application programs, system programs, machine programs, operating system software, middleware, firmware, software modules, routines, subroutines, functions, methods, procedures, software interfaces, application program interfaces, instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power level, heat tolerances, processing cycle budget, input data rates, output data rates, memory resources, data bus speeds, and other design or performance constraints.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still cooperate or interact with each other.

The various embodiments disclosed herein can be implemented in various forms of hardware, software, firmware, and/or special purpose processors. For example, in one embodiment at least one non-transitory computer readable storage medium has instructions encoded thereon that, when executed by one or more processors, cause one or more of the methodologies disclosed herein to be implemented. The instructions can be encoded using a suitable programming language, such as C, C++, object-oriented C, Java, JavaScript, Visual Basic .NET, Beginner's All-Purpose Symbolic Instruction Code (BASIC), or alternatively, using custom or proprietary instruction sets. The instructions can be provided in the form of one or more computer software applications and/or applets that are tangibly embodied on a memory device, and that can be executed by a computer having any suitable architecture. In one embodiment, the system can be hosted on a given website and implemented, for example, using JavaScript or another suitable browser-based technology. For instance, in certain embodiments, the system may leverage processing resources provided by a remote computer system accessible via network 794. The computer software applications disclosed herein may include any number of different modules, sub-modules, or other components of distinct functionality, and can provide information to, or receive information from, still other components. These modules can be used, for example, to communicate with input and/or output devices such as a display screen, a touch sensitive surface, a printer, and/or any other suitable device. Other componentry and functionality not reflected in the illustrations will be apparent in light of this disclosure, and it will be appreciated that other embodiments are not limited to any particular hardware or software configuration. Thus, in other embodiments platform 700 may comprise additional, fewer, or alternative subcomponents as compared to those included in the example embodiment of FIG. 7.

The aforementioned non-transitory computer readable medium may be any suitable medium for storing digital information, such as a hard drive, a server, a flash memory, and/or random-access memory (RAM), or a combination of memories. In alternative embodiments, the components and/or modules disclosed herein can be implemented with hardware, including gate level logic such as a field-programmable gate array (FPGA), or alternatively, a purpose-built semiconductor such as an application-specific integrated circuit (ASIC). Still other embodiments may be implemented with a microcontroller having a number of input/output ports for receiving and outputting data, and a number of embedded routines for carrying out the various functionalities disclosed herein. It will be apparent that any suitable combination of hardware, software, and firmware can be used, and that other embodiments are not limited to any particular system architecture.

Some embodiments may be implemented, for example, using a machine readable medium or article which may store an instruction or a set of instructions that, if executed by a machine, may cause the machine to perform a method, process, and/or operations in accordance with the embodiments. Such a machine may include, for example, any suitable processing platform, computing platform, computing device, processing device, computing system, processing system, computer, process, or the like, and may be implemented using any suitable combination of hardware and/or software. The machine readable medium or article may include, for example, any suitable type of memory unit, memory device, memory article, memory medium, storage device, storage article, storage medium, and/or storage unit, such as memory, removable or non-removable media, erasable or non-erasable media, writeable or rewriteable media, digital or analog media, hard disk, floppy disk, compact disk read only memory (CD-ROM), compact disk recordable (CD-R) memory, compact disk rewriteable (CD-RW) memory, optical disk, magnetic media, magneto-optical media, removable memory cards or disks, various types of digital versatile disk (DVD), a tape, a cassette, or the like. The instructions may include any suitable type of code, such as source code, compiled code, interpreted code, executable code, static code, dynamic code, encrypted code, and the like, implemented using any suitable high level, low level, object oriented, visual, compiled, and/or interpreted programming language.

Unless specifically stated otherwise, it may be appreciated that terms such as “processing,” “computing,” “calculating,” “determining,” or the like refer to the action and/or process of a computer or computing system, or similar electronic computing device, that manipulates and/or transforms data represented as physical quantities (for example, electronic) within the registers and/or memory units of the computer system into other data similarly represented as physical entities within the registers, memory units, or other such information storage transmission or displays of the computer system. The embodiments are not limited in this context.

The terms “circuit” or “circuitry,” as used in any embodiment herein, are functional and may comprise, for example, singly or in any combination, hardwired circuitry, programmable circuitry such as computer processors comprising one or more individual instruction processing cores, state machine circuitry, and/or firmware that stores instructions executed by programmable circuitry. The circuitry may include a processor and/or controller configured to execute one or more instructions to perform one or more operations described herein. The instructions may be embodied as, for example, an application, software, firmware, etc. configured to cause the circuitry to perform any of the aforementioned operations. Software may be embodied as a software package, code, instructions, instruction sets and/or data recorded on a computer-readable storage device. Software may be embodied or implemented to include any number of processes, and processes, in turn, may be embodied or implemented to include any number of threads, etc., in a hierarchical fashion. Firmware may be embodied as code, instructions or instruction sets and/or data that are hard-coded (e.g., nonvolatile) in memory devices. The circuitry may, collectively or individually, be embodied as circuitry that forms part of a larger system, for example, an integrated circuit (IC), an application-specific integrated circuit (ASIC), a system-on-a-chip (SoC), desktop computers, laptop computers, tablet computers, servers, smartphones, etc. Other embodiments may be implemented as software executed by a programmable control device. In such cases, the terms “circuit” or “circuitry” are intended to include a combination of software and hardware such as a programmable control device or a processor capable of executing the software. As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Numerous specific details have been set forth herein to provide a thorough understanding of the embodiments. It will be understood by an ordinarily skilled artisan, however, that the embodiments may be practiced without these specific details. In other instances, well known operations, components and circuits have not been described in detail so as not to obscure the embodiments. It can be appreciated that the specific structural and functional details disclosed herein may be representative and do not necessarily limit the scope of the embodiments. In addition, although the subject matter has been described in language specific to structural features and/or methodological acts, it is to be understood that the subject matter defined in the appended claims is not necessarily limited to the specific features or acts described herein. Rather, the specific features and acts described herein are disclosed as example forms of implementing the claims.

### FURTHER EXAMPLE EMBODIMENTS

The following examples pertain to further embodiments, from which numerous permutations and configurations will be apparent.

Example 1 is a system for dispensing pills, the system comprising: a base unit including a receptacle to receive a pill vial; a locking mechanism coupled to the base unit, the locking mechanism to secure the pill vial in the base unit until a release condition is satisfied; an optical scanner to read a label on the pill vial to record a prescription identifier and a dosage schedule, and to register the prescription for usage tracking; a usage tracker to monitor patient usage of the prescription based on a count of removals of the pill vial from the receptacle and a timing of the removals; and an alerting system to alert the patient of a missed dose based on the dosage schedule and on expiration of a timer configured to measure an elapsed time since a previous removal of the pill vial from the receptacle.

Example 2 includes the subject matter of Example 1, wherein the optical scanner is further to read the label on the pill vial, after re-insertion of the pill vial into the receptacle, to recognize and verify the medication based on the registration.

Example 3 includes the subject matter of Examples 1 or 2, wherein the release condition includes expiration of the timer.

Example 4 includes the subject matter of any of Examples 1-3, wherein the release condition includes one or more of verification of a personal identification number entered by a user of the system, verification of a fingerprint scan of the user of the system, and facial recognition of the user of the system.

Example 5 includes the subject matter of any of Examples 1-4, further comprising a network interface, and wherein the alerting system is further to provide an alert to a pharmacy through the network interface, the alert indicating that a refill of the prescription is needed based on the monitored patient usage and the dosage schedule.

Example 6 includes the subject matter of any of Examples 1-5, wherein the alerting system is further to provide an alert to a designated party in response to detection of abnormal medication usage based on the monitored patient usage and the dosage schedule.

Example 7 includes the subject matter of any of Examples 1-6, further comprising a user interface to display one or more of an indication of the contents of the receptacle, a history of the monitored patient usage, a time remaining until a next scheduled dose, a time remaining until a next refill.

Example 8 is a method for dispensing pills, the method comprising: engaging a locking mechanism to secure a pill vial in a base unit of a pill dispensing device until a release condition is satisfied, the pill vial received into a receptacle of the base unit; scanning a label on the pill vial to record a prescription identifier and a dosage schedule, and to register the prescription for usage tracking; monitoring patient usage of the prescription based on a count of removals of the pill vial from the receptacle and a timing of the removals; and alerting the patient of a missed dose based on the dosage schedule and on detection of an elapsed time since a previous removal of the pill vial from the receptacle.

Example 9 includes the subject matter of Example 8, further comprising scanning the label on the pill vial, after re-insertion of the pill vial into the receptacle, to recognize and verify the medication based on the registration.

Example 10 includes the subject matter of Examples 8 or 9, wherein the release condition includes detection of the elapsed time.

Example 11 includes the subject matter of any of Examples 8-10, wherein the release condition includes one or more of verification of a personal identification number entered by a user of the system, verification of a fingerprint scan of the user of the system, and facial recognition of the user of the system.

Example 12 includes the subject matter of any of Examples 8-11, further comprising alerting a pharmacy that a refill of the prescription is needed based on the monitored patient usage and the dosage schedule.

Example 13 includes the subject matter of any of Examples 8-12, further comprising alerting a designated party in response to detection of abnormal medication usage based on the monitored patient usage and the dosage schedule.

Example 14 includes the subject matter of any of Examples 8-13, further comprising displaying one or more of an indication of the contents of the receptacle, a history of the monitored patient usage, a time remaining until a next scheduled dose, a time remaining until a next refill.

Example 15 is at least one non-transitory machine-readable storage medium having instructions encoded thereon that, when executed by one or more processors, cause a process to be carried out for dispensing pills, the process comprising: engaging a locking mechanism to secure a pill vial in a base unit of a pill dispensing device until a release condition is satisfied, the pill vial received into a receptacle of the base unit; scanning a label on the pill vial to record a prescription identifier and a dosage schedule, and to register the prescription for usage tracking; monitoring patient usage of the prescription based on a count of removals of the pill vial from the receptacle and a timing of the removals; and alerting the patient of a missed dose based on the dosage schedule and on detection of an elapsed time since a previous removal of the pill vial from the receptacle.

Example 16 includes the subject matter of Example 15, further comprising scanning the label on the pill vial, after re-insertion of the pill vial into the receptacle, to recognize and verify the medication based on the registration.

Example 17 includes the subject matter of Examples 15 or 16, wherein the release condition includes detection of the elapsed time.

Example 18 includes the subject matter of any of Examples 15-17, wherein the release condition includes one or more of verification of a personal identification number entered by a user of the system, verification of a fingerprint scan of the user of the system, and facial recognition of the user of the system.

Example 19 includes the subject matter of any of Examples 15-18, further comprising alerting a pharmacy that a refill of the prescription is needed based on the monitored patient usage and the dosage schedule.

Example 20 includes the subject matter of any of Examples 15-19, further comprising alerting a designated party in response to detection of abnormal medication usage based on the monitored patient usage and the dosage schedule.

Example 21 includes the subject matter of any of Examples 15-20, further comprising displaying one or more of an indication of the contents of the receptacle, a history of the monitored patient usage, a time remaining until a next scheduled dose, a time remaining until a next refill.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications. It is intended that the scope of the present disclosure be limited not by this detailed description, but rather by the claims appended hereto. Future filed applications claiming priority to this application may claim the disclosed subject matter in a different manner and may generally include any set of one or more elements as variously disclosed or otherwise demonstrated herein.

What is claimed is:

1. A system for dispensing pills, the system comprising:
a base unit including a receptacle to receive a pill vial;
a locking mechanism coupled to the base unit, the locking mechanism to secure the pill vial in the base unit until a release condition is satisfied;
an optical scanner to read a label on the pill vial to record a prescription identifier and a dosage schedule, and to register the prescription for usage tracking;
a usage tracker to monitor patient usage of the prescription based on a count of removals of the pill vial from the receptacle and a timing of the removals; and
an alerting system to alert the patient of a missed dose based on the dosage schedule and on expiration of a timer configured to measure an elapsed time since a previous removal of the pill vial from the receptacle.

2. The system of claim 1, wherein the optical scanner is further to read the label on the pill vial, after re-insertion of the pill vial into the receptacle, to recognize and verify the medication based on the registration.

3. The system of claim 1, wherein the release condition includes expiration of the timer.

4. The system of claim 1, wherein the release condition includes one or more of verification of a personal identification number entered by a user of the system, verification of a fingerprint scan of the user of the system, and facial recognition of the user of the system.

5. The system of claim 1, further comprising a network interface, and wherein the alerting system is further to provide an alert to a pharmacy through the network interface, the alert indicating that a refill of the prescription is needed based on the monitored patient usage and the dosage schedule.

6. The system of claim 1, wherein the alerting system is further to provide an alert to a designated party in response to detection of abnormal medication usage based on the monitored patient usage and the dosage schedule.

7. The system of claim 1, further comprising a user interface to display one or more of an indication of the contents of the receptacle, a history of the monitored patient usage, a time remaining until a next scheduled dose, a time remaining until a next refill.

8. A method for dispensing pills, the method comprising:
engaging a locking mechanism to secure a pill vial in a base unit of a pill dispensing device until a release condition is satisfied, the pill vial received into a receptacle of the base unit;
scanning a label on the pill vial to record a prescription identifier and a dosage schedule, and to register the prescription for usage tracking;
monitoring patient usage of the prescription based on a count of removals of the pill vial from the receptacle and a timing of the removals; and
alerting the patient of a missed dose based on the dosage schedule and on detection of an elapsed time since a previous removal of the pill vial from the receptacle.

9. The method of claim 8, further comprising scanning the label on the pill vial, after re-insertion of the pill vial into the receptacle, to recognize and verify the medication based on the registration.

10. The method of claim 8, wherein the release condition includes detection of the elapsed time.

11. The method of claim 8, wherein the release condition includes one or more of verification of a personal identification number entered by a user of the system, verification of a fingerprint scan of the user of the system, and facial recognition of the user of the system.

12. The method of claim 8, further comprising alerting a pharmacy that a refill of the prescription is needed based on the monitored patient usage and the dosage schedule.

13. The method of claim 8, further comprising alerting a designated party in response to detection of abnormal medication usage based on the monitored patient usage and the dosage schedule.

14. The method of claim 8, further comprising displaying one or more of an indication of the contents of the receptacle, a history of the monitored patient usage, a time remaining until a next scheduled dose, a time remaining until a next refill.

15. At least one non-transitory machine-readable storage medium having instructions encoded thereon that, when executed by one or more processors, cause a process to be carried out for dispensing pills, the process comprising:
engaging a locking mechanism to secure a pill vial in a base unit of a pill dispensing device until a release condition is satisfied, the pill vial received into a receptacle of the base unit;
scanning a label on the pill vial to record a prescription identifier and a dosage schedule, and to register the prescription for usage tracking;
monitoring patient usage of the prescription based on a count of removals of the pill vial from the receptacle and a timing of the removals; and
alerting the patient of a missed dose based on the dosage schedule and on detection of an elapsed time since a previous removal of the pill vial from the receptacle.

16. The computer readable storage medium of claim 15, further comprising scanning the label on the pill vial, after re-insertion of the pill vial into the receptacle, to recognize and verify the medication based on the registration.

17. The computer readable storage medium of claim 15, wherein the release condition includes detection of the elapsed time.

18. The computer readable storage medium of claim 15, wherein the release condition includes one or more of verification of a personal identification number entered by a user of the system, verification of a fingerprint scan of the user of the system, and facial recognition of the user of the system.

19. The computer readable storage medium of claim 15, further comprising alerting a pharmacy that a refill of the prescription is needed based on the monitored patient usage and the dosage schedule.

20. The computer readable storage medium of claim 15, further comprising alerting a designated party in response to detection of abnormal medication usage based on the monitored patient usage and the dosage schedule.

21. The computer readable storage medium of claim 15, further comprising displaying one or more of an indication of the contents of the receptacle, a history of the monitored patient usage, a time remaining until a next scheduled dose, a time remaining until a next refill.
